(19) United States
(12) Patent Application Publication
(10) Pub. No.: US 2009/0264907 A1
(43) Pub. Date: Oct. 22, 2009

(54) MEDICAL DEVICE FOR CROSSING AN OCCLUDED BLOOD VESSEL

(75) Inventors: Anthony C. Vrba, Maple Grove, MN (US); Todd P. Messal, Plymouth, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., MAPLE GROVE, MN (US)

(21) Appl. No.: 12/106,032

(22) Filed: Apr. 18, 2008

(51) Int. Cl. A61B 17/22

(52) U.S. Cl. 606/159

(57) ABSTRACT

Medical devices and methods for making and using the same. An example medical device includes a tubular shaft and a crossing member disposed within the tubular shaft. The crossing member may include a loop portion. The methods for using the medical device may include advancing the medical device through the vasculature to a position where at least a portion of the device contacts an intravascular lesion and expanding the loop portion of the crossing device to displace the occlusion.

### FIELD OF THE INVENTION

The present invention pertains to medical devices, and methods for manufacturing medical devices. More particularly, the present invention pertains to elongated medical devices including an elongated tubular shaft and a crossing member, and to methods for manufacturing and using such devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

The invention provides design, material, manufacturing method, and use alternatives for medical devices. An example medical device includes a tubular shaft and a crossing member disposed within the tubular shaft. The crossing member may include a loop portion. The methods for using the medical device may include advancing the medical device through the vasculature to a position where at least a portion of the device contacts a vascular blockage and expanding the loop portion of the crossing device to displace the vascular blockage.

The above summary of some embodiments is not intended to describe each disclosed embodiment or every implementation of the present invention. The Figures, and Detailed Description, which follow, more particularly exemplify these embodiments

### BRIEF DESCRIPTION OF THE DRAWINGS

The invention may be more completely understood in consideration of the following detailed description of various embodiments of the invention in connection with the accompanying drawings, in which:

FIG. 1 is a partial cross-sectional side view of an example medical device;

FIG. 2 is a partial cross-sectional side view of the device depicted in FIG. 1 where a crossing member extends distally from the distal end of a tubular shaft;

FIG. 3A is a partial cross-sectional side view of the device depicted in FIGS. 1 and 2 in contact with an occlusion;

FIG. 3B is a partial cross-sectional side view of the device depicted in FIGS. 1 and 2 where a loop portion of the device is expanded;

FIG. 3C is a partial cross-sectional side view of the device depicted in FIGS. 1 and 2 navigated through an occlusion;

FIG. 4 is a cross-sectional view taken through line 4-4;

FIG. 5 is an alternative cross-sectional view taken through line 4-4;

FIG. 6 is an alternative cross-sectional view taken through line 4-4;

FIG. 7 is an alternative cross-sectional view taken through line 4-4;

FIG. 8 is a perspective view of another example crossing member;

FIG. 9 is a perspective view of another example crossing member;

FIG. 10 is a partial cross-sectional view of another example medical device; and

FIG. 11 is a partial cross-sectional view of another example medical device.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

Within the vasculature of a patient, a blockage such as a lesion, stenosis, occlusion, and/or the like may, along with potentially posing a number of other risks, define or otherwise create a formidable barrier to a medical device (e.g., a catheter, guidewire, etc.) attempting to pass therethrough during a medical intervention. This may be further complicated when the occlusion is “total” (i.e., as in a “chronic total occlusion”) and/or when the occlusion is calcified. Because of this issue, a number of medical devices have be concocted for crossing occlusions. These devices, sometimes called “crossing wires” or “crossing devices”, generally include material and design consideration that are directed at improving the chances of navigating it or other medical device successfully past or otherwise through a lesion.

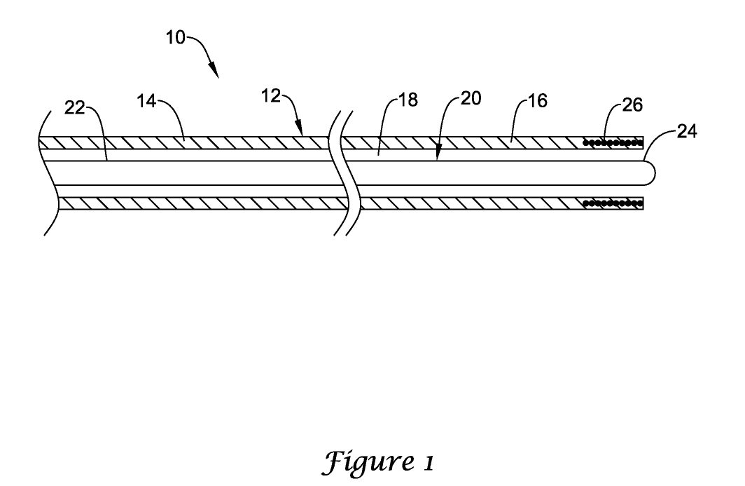

FIG. 1 illustrates an example crossing device 10. Device 10 may include an elongate tubular shaft 12. Shaft 12 includes a proximal portion 14, a distal portion 16, and a lumen 18 extending at least partially the length therethrough. A crossing member 20 may be disposed in lumen 18. Crossing member 20 may include a proximal or body portion 22 and a distal or loop portion 24. Shaft 12 and/or crossing member 20 may be configured to contact an intravascular occlusion and displace a portion of the occlusion. For example, device 10 may be advanced through a blood vessel to a position where the shaft 12, crossing member 20, or both are in contact with an intravascular occlusion. Once positioned, shaft 12 may be shifted proximally relative to crossing member 20 and loop portion 24 may expand and displace at least a portion of the occlusion. After one or more these “cycles” (i.e., where shaft 12 and/or crossing member 20 engages an occlusion and crossing member 20 displaces a portion of the occlusion), a large enough opening may be formed in the occlusion so that device 10 may advanced through the occlusion. Once past the occlusion, device 10 may allow other devices to similarly pass the occlusion by passing through (e.g., through lumen 18) or over shaft 12.

Tubular shaft 12 may have a variety of forms and/or configurations. Some examples of example materials used for constructing shaft 12 and configurations of shaft 12 are discussed in more detail below. In at least some embodiments, tubular shaft 12 may resemble a catheter and, consequently, it may include any of the features and/or characteristics of typical catheters. Moreover, shaft 12 may include one or more additional features that distinguishes it from typical catheters. For example, distal portion 16 of shaft 12 may include a stiffened region or stiffening member 26. Although stiffening member 26 is only shown in FIG. 1 as being a component of shaft 12, it can be appreciated than any of the shafts disclosed herein may include a stiffening member 26. Stiffening member 26 may take the form of a braid, coil, mesh, or like or it may comprise a generally stiffer material such a relatively stiff polymer or metal. The inclusion of stiffening member 26 may improve the ability of tubular shaft 12 to contact an occlusion and, in at least some embodiments, to bump or “ram” into the occlusion in a manner such that a portion of shaft 12 embeds within the occlusion while maintaining the integrity of distal portion 16. Stiffening member 26 may also provide shaft 12 with a number of additional desirable features. Furthermore, distal portion 16 of shaft 12 may include a thinned or “sharpened” distal end (not shown) that further improves the ability of shaft 12 to embed into and break up an occlusion.

Crossing member 20 may similarly have a number of different forms and/or configurations. Some specific examples of alternative configurations for crossing member 20 are discussed in more detail below including some of the appropriate materials that may be used in constructing crossing member 20. In general, crossing member 20 may take the form of a wire or shaft that extends down the length of tubular shaft 12 from proximal portion 14 to distal portion 16, loops around at loop portion 24, and then extends back down the length of tubular shaft 12 from distal portion 16 to proximal portion 14. In at least some embodiments, one or both of the ends of the wire forming crossing member 20 may be accessible to a clinician at the proximal end of tubular shaft 12. This allows the clinician to control the position and/or orientation of crossing member 20 during use of device 10.

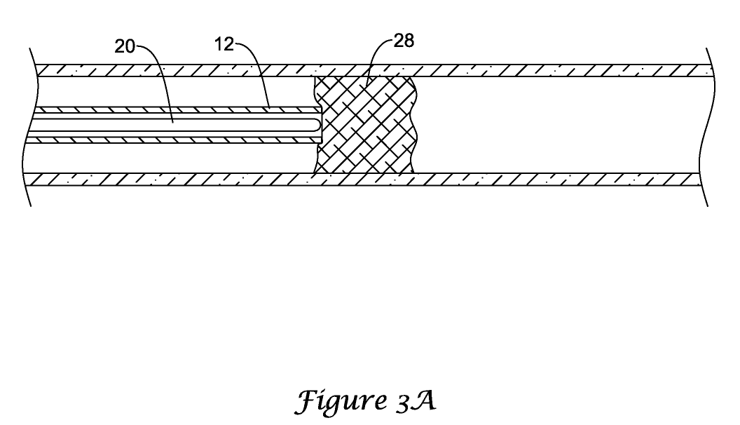

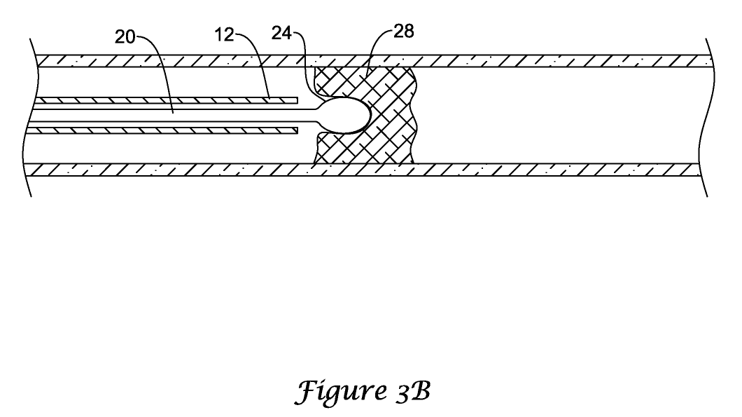

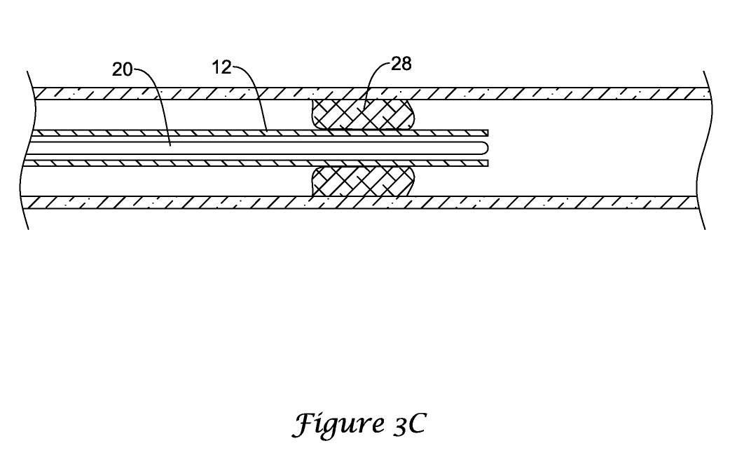

As suggested above, the method for using device 10 may include a number of steps including, for example, those illustrated in FIGS. 3A-3C. For example, after providing the appropriate embodiment of device 10 for a particular intervention, device 10 can be advanced through a body lumen (e.g., a blood vessel) to a position adjacent a target site (e.g., an intravascular occlusion). The advancing step may result in tubular shaft 12, crossing member 20, or both being in contact with the intravascular occlusion as shown in FIG. 3A. Being brought into contact with the intravascular occlusion may be understood to being any level of contact including minimal contact (e.g., “barely touching”), being embedded within the occlusion (e.g., where a portion of shaft 12, crossing member 20, or both are embedded in the occlusion), or any level in-between. Embedding shaft 12 and/or crossing member 20 within the occlusion may help to begin (or, ultimately, complete) the process of breaking the occlusion by altering the occlusion, moving a portion of the occlusion, and/or deforming the occlusion such that device 10 and/or other devices can navigate past the occlusion.

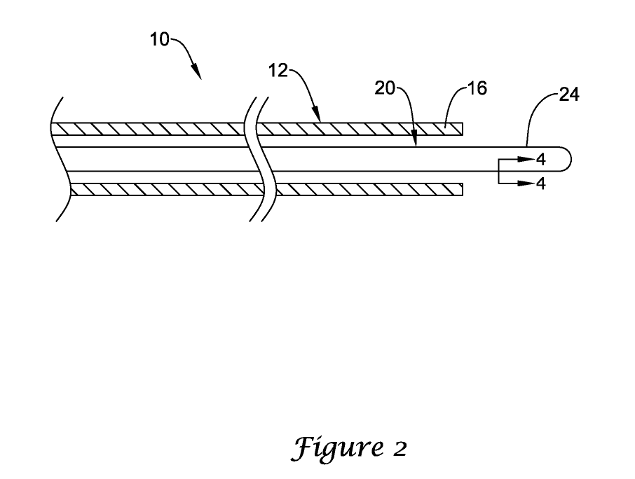

While advancing, crossing device 20 is generally disposed within shaft 12 (e.g., within lumen 18 of shaft 12) such that loop portion 24 is completely contained within lumen 18. Alternatively, loop portion 24, any section of loop portion 24, or any portion of crossing member 20, may extend distally out from shaft 12 while advancing device 10 through the vasculature or at any suitable time during the intervention as shown in FIG. 2. This later embodiment may provide device 10 with a rounded distal end that is generally atraumatic and/or allow crossing member 20 (rather than shaft 12) to contact the occlusion, if such an arrangement is desired. It can be appreciated that that position of crossing member 20 relative to shaft 12 may vary considerable including variations that occur during the intervention as crossing member 20 may be axially moveable and/or rotatably movable within shaft 12.

Once tubular shaft 12, crossing member 20, or both are in contact with the intravascular occlusion, shaft 12 may be shifted proximally relative crossing member 20. This step may be optional, however, depending on the circumstances of the intervention. Proximally shifting shaft 12 relative to crossing member 20 may include distally advancing crossing member 20 (while proximally retracting shaft 12 or while holding shaft 12 stationary), proximally retracting shaft 12 (while distally advancing crossing member 20 or while holding crossing member 20 stationary), or both.

Generally the next step may include expanding loop portion 24 of crossing member 20 as shown in FIG. 3B. Expanding loop portion 24 may occur in a number of different ways. For example, expanding loop portion 24 may occur when crossing member 20 is distally advanced into an occlusion (which is marked with reference number 28 in FIG. 3B) such that loop portion 24 flares radially outward into a loop configuration in response to encountering axial resistance. This may or may not occur as part of or in conjunction with the “shifting” step described above. In some of these and other embodiments, expanding loop portion 24 may occur due at least in part to the material composition of loop portion 24. For example, loop portion 24 may include a shape memory material such as nickel-titanium alloy such that loop portion 24 expands when unconstrained (e.g., when loop portion 24 is disposed outside of lumen 18) and exposed to the appropriate temperature conditions (e.g., when “set” to assume the expanded shape at or near body temperature). In some of these and/or other embodiments, loop portion 24 may expand when being distally advanced until encountering resistance (e.g., when hitting occlusion 28) such that additional longitudinal force on crossing member 20 causes loop portion 24 to flare outward.

Expanding loop portion 24 may displace a portion of the occlusion. For example, as loop portion 24 expands into the loop configuration, the loop portion 24 may press against the occlusion and displace a portion of it much like how a pastry blender might contact and break up a pastry mixture. This “displacing effect” may be accentuated by rotating crossing member 20 so that loop portion 24 can act like an egg beater to break up the occlusion.

Upon completion of the previously discussed steps, the clinician may evaluate the occlusion to determine whether or not it is passable (e.g., using suitable imaging techniques). If the occlusion is deemed passable, device 10 and/or other suitable devices may be navigated through the occlusion as illustrated in FIG. 3C. If the occlusion is not deemed passable, any or all of the method steps may be repeated one or more times until the occlusion is sufficiently displaced so as to be passable.

As alluded to above, device 10 and the various components thereof, may vary in configuration and/or material composition. In general, device 10 and the components thereof may include suitable materials such as metals, polymer, metal-polymer composites, combinations thereof, and the like, or any other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

As alluded to above, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2-0.44% strain before plastically deforming.

In some embodiments, the linear elastic and/or non-super-elastic nickel-titanium alloy is an alloy that does not show any martensite/austenite phase changes that are detectable by DSC and DMTA analysis over a large temperature range. For example, in some embodiments, there may be no martensite/austenite phase changes detectable by DSC and DMTA analysis in the range of about −60° C. to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties and has essentially no yield point.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of device 10 (including any or all of the components thereof) may also be doped with, made of, or otherwise include a radiopaque material. Radiopaque materials are understood to be materials capable of producing a relatively bright image on a fluoroscopy screen or another imaging technique during a medical procedure. This relatively bright image aids the user of device 10 in determining its location. Some examples of radiopaque materials can include, but are not limited to, gold, platinum, palladium, tantalum, tungsten alloy, polymer material loaded with a radiopaque filler, and the like. Additionally, radiopaque marker bands and/or coils may be incorporated into the design of device 10 to achieve the same result.

In some embodiments, a degree of MRI compatibility is imparted into device 10. For example, to enhance compatibility with Magnetic Resonance Imaging (MRI) machines, it may be desirable to make device 10 (including any or all of the components thereof) in a manner that would impart a degree of MRI compatibility. For example, device 10 or portions thereof may be made of a material that does not substantially distort the image and create substantial artifacts (artifacts are gaps in the image). Certain ferromagnetic materials, for example, may not be suitable because they may create artifacts in an MRI image. Device 10 or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

As indicated above, device 10 may also include one or more different polymers. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®)), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

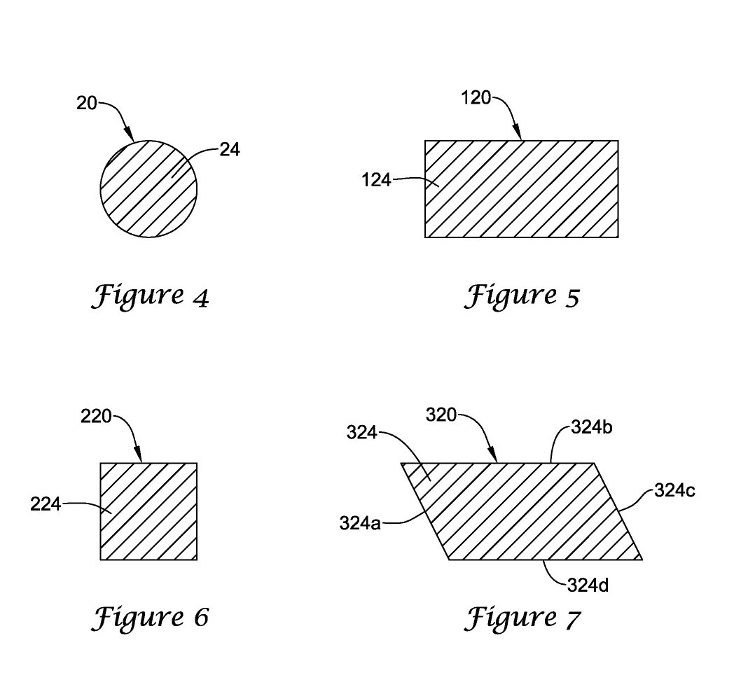

In addition to material difference, other variations are contemplated for device 10. For example, in at least some embodiments, crossing member 20 may include at least a region (e.g., loop portion 20) that has a generally circular cross-sectional shape as depicted in FIG. 4. However, this is not intended to be limiting. For example, FIG. 5 illustrates another example crossing member 120 (which may be similar in form and function to other crossing members disclosed herein) that has a loop portion 124 with a generally rectangular cross-sectional shape. Similarly, FIG. 6 illustrates another example crossing member 220 (which may be similar in form and function to other crossing members disclosed herein) that has a loop portion 224 with a generally square cross-sectional shape and FIG. 7 illustrates another example crossing member 320 (which may be similar in form and function to other crossing members disclosed herein) that has a loop portion 324 with a cross-sectional shape substantially resembling a parallelogram. It can be appreciated that numerous other shapes are contemplated. Furthermore, the various embodiments of crossing members may or may not have the same cross-sectional shape along the entire length thereof. Additionally, the various embodiments may include crossing members that include loop portions with non-continuous segments and/or materials. Any of these or other crossing members may be used in conjunction with any of the shaft disclosed herein.

The parallelogram-shaped crossing member 320 may also include additional structural features. For example, any one or more of the sides 324a/324b/324c/324d of parallelogram-shaped crossing member 320 may be form a cutting or blade-like surface that may increase the ability of crossing member 320 to cut into and break up an occlusion. Other embodiments of crossing members may include similar structural features. Any of these or other crossing members may be used in conjunction with any of the shaft disclosed herein.

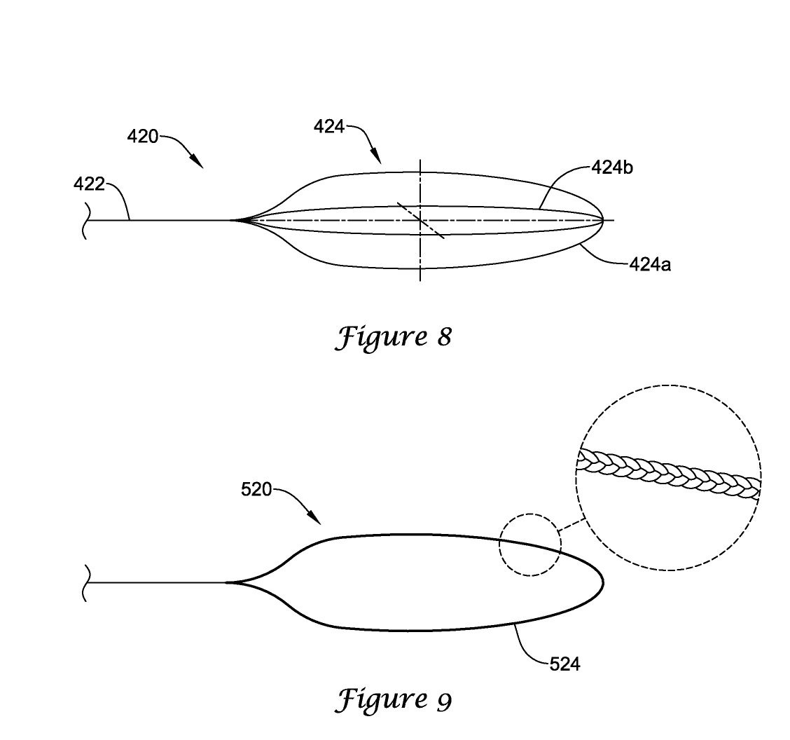

Other embodiments of crossing members are contemplated that include loop portions with one or more additional loops and/or different body portions. For example, FIG. 8 illustrates an example crossing member 420 (which may be similar in form and function to other crossing members disclosed herein) that includes a loop portion 424 with two loops 424a/424b. Loops 424a/424b may be oriented generally orthogonal relative to one another (as shown) or they may be oriented in any other suitable arrangement. Other embodiments are contemplated that include three, four, five, six, or more loops. Any of these or other crossing members may be used in conjunction with any of the shaft disclosed herein.

Crossing member 420 may also include a body portion 422 that comprises a generally singular shaft that is defined by merging the loops 424a/424b of loop portion 424 into a shaft, by attaching a shaft to loop portion 424, or in any other suitable manner. It can be appreciated that any of the other crossing members disclosed herein may include body portions that resemble body portion 422. Any of these or other crossing members may be used in conjunction with any of the shaft disclosed herein.

Another example crossing member 520 (which may be similar in form and function to other crossing members disclosed herein) is depicted in FIG. 9. Crossing member 520 may include a loop portion 524 that includes a braid. The braid may include two, three, four, or more wires that are braided together. In some embodiments, the wires forming the braid are all made of the same material. In other embodiments, some of the wires are formed of a different material. The materials forming the braid may include any of those disclosed herein including radiopaque materials. Any of these or other crossing members may be used in conjunction with any of the shaft disclosed herein.

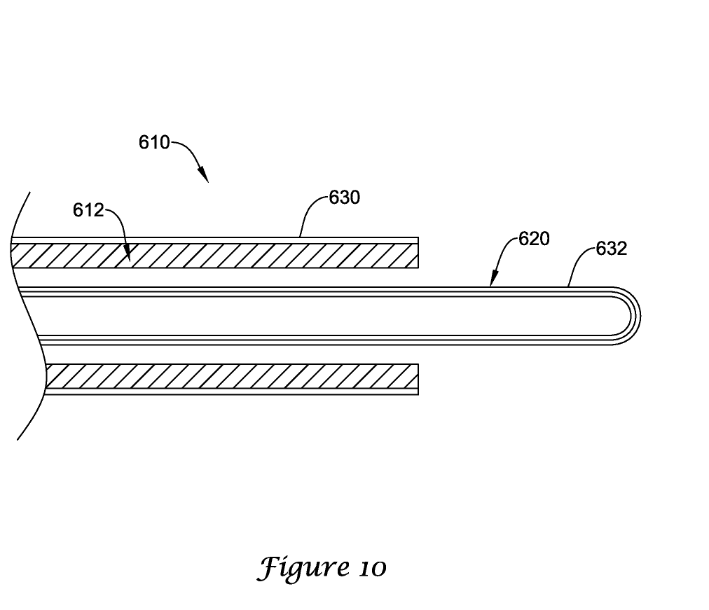

Turning now to FIG. 10, another example device 610 is illustrated that may be similar in form and function to any of the other devices or device components disclosed herein. Device 610 includes tubular shaft 612 and crossing member 620. In this embodiment, one or both of shaft 612 and crossing member 620 may include a coating. For example, shaft 612 may include a coating 630 along the outer surface thereof and/or crossing member 620 may include a coating 632 along the outer surface thereof.

Coating 630 and/or 632 may be a lubricious, a hydrophilic, a protective, or other type of coating. Hydrophobic coatings such as fluoropolymers provide a dry lubricity which improves device handling and exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

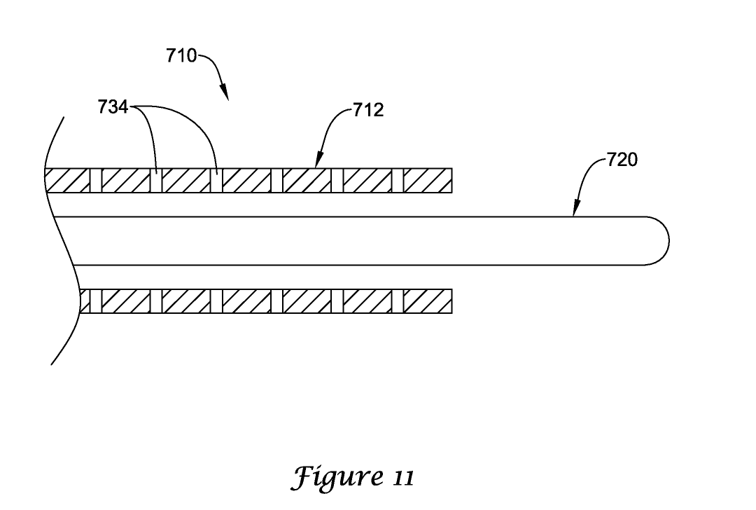

FIG. 11 illustrates another example device 710 that may be similar in form and function to any of the other devices or device components disclosed herein. Device 710 includes tubular shaft 712 and crossing member 720. Tubular shaft 712 may be made from any of the materials disclosed herein such as nickel-titanium alloy. In at least some embodiments, shaft 712 includes a plurality of cuts, apertures, and/or slots 734 formed therein. Slots 734 can be formed by methods such as micro-machining, saw-cutting (e.g., using a diamond grit embedded semiconductor dicing blade), laser cutting, electron discharge machining, grinding, milling, casting, molding, chemically etching or treating, or other known methods, and the like. In some such embodiments, the structure of the shaft 712 is formed by cutting and/or removing portions of the tube to form slots 734. Some example embodiments of appropriate micromachining methods and other cutting methods, and structures for tubular members and/or shaft including slots and medical devices including tubular members and/or shafts are disclosed in U.S. Pat. Publication Nos. US 2003/0069522 and US 2004/0181174-A2; and U.S. Pat. Nos. 6,766,720; and 6,579,246, the entire disclosures of which are herein incorporated by reference. Some example embodiments of etching processes are described in U.S. Pat. No. 5,106,455, the entire disclosure of which is herein incorporated by reference. It should be noted that the methods for manufacturing device 10 may include forming slots 734 in shaft 712 using any of these or other manufacturing steps.

Various embodiments of arrangements and configurations of slots 734 are contemplated. In some embodiments, at least some, if not all of slots 734 are disposed at the same or a similar angle with respect to the longitudinal axis of the shaft 712. As shown, slots 734 can be disposed at an angle that is perpendicular, or substantially perpendicular, and/or can be characterized as being disposed in a plane that is normal to the longitudinal axis of shaft 712. However, in other embodiments, slots 734 can be disposed at an angle that is not perpendicular, and/or can be characterized as being disposed in a plane that is not normal to the longitudinal axis of shaft 712. Additionally, a group of one or more slots 734 may be disposed at different angles relative to another group of one or more slots 734. The distribution and/or configuration of slots 734 can also include, to the extent applicable, any of those disclosed in U.S. Pat. Publication No. US 2004/0181174, the entire disclosure of which is herein incorporated by reference.

Slots 734 may be provided to enhance the flexibility of shaft 712 while still allowing for suitable torque transmission characteristics. Slots 734 may be formed such that one or more rings and/or turns interconnected by one or more segments and/or beams are formed in shaft 712, and such rings and beams may include portions of shaft 712 that remain after slots 734 are formed in the body of shaft 712. Such an interconnected ring structure may act to maintain a relatively high degree of torsional stiffness, while maintaining a desired level of lateral flexibility. In some embodiments, some adjacent slots 734 can be formed such that they include portions that overlap with each other about the circumference of shaft 712. In other embodiments, some adjacent slots 734 can be disposed such that they do not necessarily overlap with each other, but are disposed in a pattern that provides the desired degree of lateral flexibility.

Additionally, slots 734 can be arranged along the length of, or about the circumference of, shaft 712 to achieve desired properties. For example, adjacent slots 734, or groups of slots 734, can be arranged in a symmetrical pattern, such as being disposed essentially equally on opposite sides about the circumference of shaft 712, or can be rotated by an angle relative to each other about the axis of shaft 712. Additionally, adjacent slots 734, or groups of slots 734, may be equally spaced along the length of shaft 712, or can be arranged in an increasing or decreasing density pattern, or can be arranged in a non-symmetric or irregular pattern. Other characteristics, such as slot size, slot shape and/or slot angle with respect to the longitudinal axis of shaft 712, can also be varied along the length of shaft 712 in order to vary the flexibility or other properties. In other embodiments, moreover, it is contemplated that the portions of the tubular shaft, such as a proximal section, a distal section, or the entire shaft 712, may not include any such slots 734.

As suggested above, slots 734 may be formed in groups of two, three, four, five, or more slots 734, which may be located at substantially the same location along the axis of shaft 712. Within the groups of slots 734, there may be included slots 734 that are equal in size (i.e., span the same circumferential distance around shaft 712). In some of these as well as other embodiments, at least some slots 734 in a group are unequal in size (i.e., span a different circumferential distance around shaft 712). Longitudinally adjacent groups of slots 734 may have the same or different configurations. For example, some embodiments of shaft 712 include slots 734 that are equal in size in a first group and then unequally sized in an adjacent group. It can be appreciated that in groups that have two slots 734 that are equal in size, the beams (i.e., the portion of shaft 712 remaining after slots 734 are formed therein) are aligned with the center of shaft 712. Conversely, in groups that have two slots 734 that are unequal in size, the beams are offset from the center of shaft 712. Some embodiments of shaft 712 include only slots 734 that are aligned with the center of shaft 712, only slots 734 that are offset from the center of shaft 712, or slots 734 that are aligned with the center of shaft 712 in a first group and offset from the center of shaft 712 in another group. The amount of offset may vary depending on the depth (or length) of slots 734 and can include essentially any suitable distance.

Numerous other arrangements are contemplated that take advantage of the various arrangements and/or configurations discussed above.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A medical device for crossing an occlusion in a blood vessel, comprising:
an elongate tubular shaft having a proximal end, a distal end, and a lumen extending therethrough; and
a crossing member disposed in the lumen for crossing an intravascular occlusion, the crossing member including a body portion and a loop portion, the loop portion being configured to engage and displace a portion of the occlusion during an intravascular crossing procedure.

2. The medical device of claim 1, wherein the distal end of the shaft includes a stiffened region.

3. The medical device of claim 1, wherein the body portion includes a single shaft member.

4. The medical device of claim 1, wherein the body portion includes two or more shaft members.

5. The medical device of claim 1, wherein the loop portion includes a braid.

6. The medical device of claim 1, wherein the loop portion has a substantially circular cross-sectional shape.

7. The medical device of claim 1, wherein the loop portion has a non-circular cross-sectional shape.

8. The medical device of claim 6, wherein the non-circular cross-sectional shape of the loop portion includes one or more blade surfaces.

9. The medical device of claim 1, wherein the loop portion includes a single loop.

10. The medical device of claim 1, wherein the loop portion includes two or more loops.

11. The medical device of claim 1, wherein the elongate tubular shaft has a plurality of slots formed therein.

12. The medical device of claim 1, wherein the elongate tubular shaft, the crossing member, or both include a nickel-titanium alloy, stainless steel, platinum, tungsten, or combinations thereof.

13. The medical device of claim 1, wherein the elongate tubular shaft, the crossing member, or both include a coating.

14. A method for crossing an occlusion in a blood vessel, the method comprising:
providing an occlusion crossing device, the device comprising an elongate tubular shaft having a lumen defined therein and a crossing member disposed in the lumen, the crossing member including a body portion and a loop portion;
advancing the occlusion crossing device through a blood vessel to a position where the tubular shaft, the crossing member, or both are in contact with an intravascular occlusion;
shifting the tubular shaft proximally relative to the crossing member; and
expanding the loop portion of the crossing member so that the loop portion displaces at least a portion of the occlusion.

15. The method of claim 14, wherein the advancing step embeds a portion of the tubular shaft within intravascular occlusion.

16. The method of claim 14, wherein the shifting step includes proximally retracting the tubular shaft while holding the crossing member substantially stationary.

17. The method of claim 14, wherein the shifting step includes proximally retracting the tubular shaft while distally advancing the crossing member.

18. The method of claim 14, wherein the shifting step includes distally advancing the crossing member.

19. The method of claim 14, further comprising one or more additional advancing steps, shifting steps, expanding steps, or combinations thereof.

20. The method of claim 14, further comprising rotating the crossing member.

21. A method for crossing an occlusion in a blood vessel, the method comprising:
providing an occlusion crossing device, the device comprising an elongate tubular shaft having a lumen defined therein and a crossing member disposed in the lumen, the crossing member including a body portion and a loop portion;
advancing the occlusion crossing device through a blood vessel to a position where the tubular shaft contacts an intravascular occlusion;
retracting the tubular shaft proximally while advancing the crossing member distally;
expanding the loop portion of the crossing member so that the loop portion displaces at least a portion of the occlusion; and
performing one or more additional advancing steps, retracting steps, expanding steps, or combinations thereof.

22. The method of claim 21, wherein the advancing step embeds a portion of the tubular shaft within intravascular occlusion.

23. The method of claim 21, further comprising rotating the crossing member.
